(19) United States
(12) Patent Application Publication
(10) Pub. No.: US 2016/0114188 A1
(43) Pub. Date: Apr. 28, 2016

(54) METHOD AND APPARATUS FOR DETERMINING A RADIATION DOSE OF A RADIOPHARMACEUTICAL

(71) Applicant: Siemens Aktiengesellschaft, Muenchen (DE)

(72) Inventor: Sebastian Schmidt, Weisendorf (DE)

(73) Assignee: Siemens Aktiengesellschaft, Muenchen (DE)

(21) Appl. No.: 14/921,356

(22) Filed: Oct. 23, 2015

(30) Foreign Application Priority Data
Oct. 24, 2014 (DE) 102014221634.6

(51) Int. Cl. A61N 5/10; A61B 5/026; A61B 5/00

(52) U.S. Cl. CPC A61N 5/1039; A61B 5/4839; A61B 5/0263; A61N 2005/1021; A61B 2576/00

(57) ABSTRACT

In a method or system for determining a radiation dose of a radiopharmaceutical, magnetic resonance image data of an object under examination are acquired by operation of a magnetic resonance image data acquisition unit. At least one target area and/or at least one area at risk for accumulation of the radiopharmaceutical is/are segmented in the magnetic resonance image data. Molecular image data of the object under examination by operation of a molecular image data acquisition unit during the accumulation of the radiopharmaceutical in the at least one target area and/or the at least one area at risk. A radiation dose of the radiopharmaceutical is determined in the at least one target area and/or the at least one area at risk using the molecular image data.

### BACKGROUND OF THE INVENTION

1. Field of the Invention

The invention concerns a method for determining a radiation dose of a radiopharmaceutical, a radiation dose determining unit and apparatus, and a non-transitory, computer-readable storage medium encoded with programming instructions for implementing such a method.

2. Description of the Prior Art

For certain medical applications, a radiopharmaceutical is administered to an object under examination, in particular a patient. The radiopharmaceutical can be present, for example, in the form of a liquid radioisotope. The radiopharmaceutical can be injected into the object under examination. The radiopharmaceutical then typically accumulates in a desired target area, and possibly also in an undesired area at risk of the object under examination. Consequently, the radiopharmaceutical supplies a radiation dose to the target area and/or area at risk, which causes damage to tissue located in the target area and/or area at risk. For example, the radiopharmaceutical can be used to treat a thyroid carcinoma or bone metastases. Alternatively or additionally, the radiopharmaceutical can be used for diagnostic purposes.

When using the radiopharmaceutical, it is desirable to determine the radiation dose of the radiopharmaceutical in the target area and/or area at risk. This is advantageous when the distribution of the radiopharmaceutical in a body of the object under examination is unknown. This can be the case, for example, when the radiopharmaceutical is a radioisotope bound to a specific binding site such as an antibody or peptide. A precise distribution of receptors for the specific binding site in the body of the object under examination is typically not known and so it is typically not possible to calculate the distribution of the radiation dose of the radiopharmaceutical directly.

### SUMMARY OF THE INVENTION

An object of the invention is to enable improved determination of a radiation dose of a radiopharmaceutical.

The method according to the invention for determining a radiation dose of a radiopharmaceutical has the following steps. Magnetic resonance image data of an object under examination are acquired by operation of a magnetic resonance image data acquisition unit (scanner). At least one target area and/or at least one area at risk for the accumulation of the radiopharmaceutical in the magnetic resonance image data is/are segmented. Molecular image data of the object under examination are acquired by operation of a molecular image data acquisition unit during the accumulation of the radiopharmaceutical in the at least one target area and/or the at least one area at risk. A radiation dose of the radiopharmaceutical in the at least one target area and/or the at least one area at risk using the molecular image data is determined.

The radiopharmaceutical includes a radioactive substance. The radioactive substance can be designed or selected to apply a radiation dose to a target area of the object under examination. The radioactive substance can also be embodied for detection in the molecular image data. The radiopharmaceutical can additionally be radioactively labeled. In this case, the radioactive labeling is selected such that the radiopharmaceutical can be detected by the molecular image data. Various substance classes are conceivable for the actual radiopharmaceutical. The radioactive substance is then in particular coupled to one substance of the substance classes. For example, the radiopharmaceutical can comprise an antibody, an antibody fragment (for example a Fab fragment), a peptide, a hormone, a hormone analog (for example octreotide), a neurotransmitter (for example DOPA), a salt of radioactive isotopes (for example radio-chloride, sodium fluoride) or a precursor and/or a module of one of the named substances (for example L-DOPA as a precursor of DOPA or iodine for a hormone). Obviously, further substance classes are also conceivable for the radiopharmaceutical as long as they appear appropriate to those skilled in the art.

A radiation dose typically characterizes a variable describing the effect of ionizing radiation in material, typically a tissue of the object under examination. The radiation dose can be expressed, for example, as an energy dose representing energy released per mass unit to the material.

The object under examination is in particular a patient. The determined radiation dose determined can be made available as an electrical signal such that, after its determination, the radiation dose is presented as an output for a user on an output unit, for example, a display monitor. In this case, the display of the radiation dose can be spatially resolved. For example, the radiation dose can be displayed superimposed on the magnetic resonance image data and/or molecular image data. It is also conceivable for the magnetic resonance image data and molecular image data to be displayed merged and/or in registration with each other. Alternatively or additionally, for the display of the radiation dose, the provision of the radiation dose can include the storage of the radiation dose in a database following its determination.

The at least one target area can represent an anatomical structure of the object under examination, in particular an organ structure and/or a tissue structure, for example a tumor tissue. The at least one target area can represent the region in the object under examination in which the radiopharmaceutical is to accumulate. For example, the target area is the region in the object under examination in which the radiopharmaceutical should release a major part of its radiation dose, for example for therapeutic purposes. The radiopharmaceutical should accumulate in the target area such that a radiation dose of the radiopharmaceutical exceeds a threshold value in the target area. If the radiopharmaceutical is a radioisotope coupled to a specific binding site, in particular receptors for the specific binding site are localized in the target area. This enables the accumulation of the radiopharmaceutical in the target area to be ensured.

The at least one area at risk can represent an anatomical structure of the object under examination, such as an organ structure and/or a tissue structure. The at least one area at risk can represent the region in the object under examination in which accumulation of the radiopharmaceutical is not wanted. Accumulation of the radiopharmaceutical in the area at risk should be prevented to the extent that a radiation dose of the radiopharmaceutical in the area at risk is below a threshold value. The at least one area at risk for the accumulation of the radiopharmaceutical is typically characterized by being susceptible to radioactive radiation. For example, an increased radiation dose of the radiopharmaceutical can result in damage to tissue located in the area at risk. It is also conceivable for the radiopharmaceutical to accumulate in the at least one area at risk such that it is no longer possible for a sufficient accumulation of the radiopharmaceutical in the target area to take place. Typical examples of possible areas at risk in the object under examination are the liver, spleen, kidney, bladder, bone marrow, etc. Further areas at risk are known to those skilled in the art.

The target area and/or the area at risk for the accumulation of the radiopharmaceutical typically result from the pharmacological properties of the radiopharmaceutical. A typical target area and/or area at risk for a radiopharmaceutical used is usually known to those skilled in the art. In this way, those skilled in the art can use knowledge of the typical target area and/or area at risk for the radiopharmaceutical that is used, in order to segment the target area and/or area at risk in the magnetic resonance image data. If, for example, a liver metastasis is the target area for the radiopharmaceutical, the surrounding tissue will usually represent an area at risk for the radiopharmaceutical. If, for example, a bone metastasis is the target area for the radiopharmaceutical, the spleen can represent an area at risk for the radiopharmaceutical.

The accumulation of the radiopharmaceutical describes the period following the introduction of the radiopharmaceutical into the object under examination. The accumulation of the radiopharmaceutical can take place in the period in which a concentration of the radiopharmaceutical in the at least one target area and/or area at risk changes, in particular increases. The accumulation of the radiopharmaceutical can then be terminated when a concentration of the radiopharmaceutical in the at least one target area and/or area at risk reaches its maximum value and/or drops again and/or can no longer be identified in the molecular image data.

For the acquisition of the magnetic resonance image data, usually the body of the object under examination is exposed to a relatively high basic magnetic field produced by a basic field magnet. Additionally, gradient circuits are activated with a gradient coil unit. A radio-frequency antenna unit then emits radio-frequency pulses, in particular excitation pulses, by suitable antenna units, which cause nuclear spins of specific atoms excited to resonance by these radio-frequency pulses to be flipped by a defined flip angle relative to the magnetic field lines of the basic magnetic field. Upon relaxation of the nuclear spins, radio-frequency signals, so-called magnetic resonance signals, are radiated and are received by suitable radio-frequency antennas and then processed further. The desired magnetic resonance image data are reconstructed from the raw data acquired in this manner. A specific measurement, therefore, requires the emission of a specific magnetic resonance sequence, also known as a pulse sequence, composed of a series of radio-frequency pulses, in particular excitation pulses and refocusing pulses, and gradient pulses that are emitted in coordination thereto in different gradient axes along different spatial directions. Chronologically matching readout windows specifying the period in which the induced magnetic resonance signals are acquired are set. In this case, the acquisition of the magnetic resonance image data takes place from an area to be examined, also called the recording volume (field of view), encompassing the at least one target area and/or area at risk.

The acquisition of the magnetic resonance image data takes place such that the at least one target area and/or area at risk can be demarcated particularly well from surrounding tissue. In this way, as described below, a magnetic resonance sequence that is particularly suitable for depicting the at least one target area and/or area at risk can be used for the acquisition of the magnetic resonance image data. The at least one target area and/or the at least one area at risk can then be segmented in the magnetic resonance image data. In this case, the segmentation can be performed manually by a user and/or automatically, for example by execution of a threshold-based and/or atlas-based algorithm.

The acquisition of the molecular image data of the object under examination can include an acquisition of nuclear image data and/or functional image data. In this case, molecular image data typically depicts molecular and/or biochemical processes in the body of the object under examination. Unlike the magnetic resonance image data, which depicts the anatomy of the object under examination and enables segmentation of the at least one target area and/or area at risk, the molecular image data is suitable for determining a distribution of the radiopharmaceutical in the object under examination. The molecular image data acquisition unit can be, for example, a positron emission tomography (PET) image data acquisition unit (scanner) or a single photon emission tomography (SPECT) image data acquisition unit (scanner). In this case, the acquisition of the molecular image data takes place from an area under examination encompassing the at least one target area and/or area at risk.

The acquisition of the molecular image data is performed following the introduction, for example oral administration and/or injection, of the radiopharmaceutical into the object under examination. In this case, the acquisition of the molecular image data begins immediately after the start of the introduction of the radiopharmaceutical. The molecular image data then can be acquired over a continuous period and follow the course of the accumulation of the radiopharmaceutical from the beginning. With the continuous introduction of the radiopharmaceutical over a defined determined period, the acquisition of the molecular image data can take place at least over a part of the period determined. For example, the radiation dose determined using the molecular image data can be used to adjust the introduction of the radiopharmaceutical, as will be described below. The acquisition of the molecular image data is time-resolved or dynamic. This results in the molecular image data being able to describe a temporal course of the accumulation of the radiopharmaceutical in the at least one target area and/or area at risk. In this way, the acquisition of the molecular image data can include the acquisition of several temporally successive molecular single images. The several molecular single images can then depict a course of the accumulation of the radiopharmaceutical in the at least one target area and/or area at risk. The several molecular single images can furthermore be acquired at various times during the introduction of the radiopharmaceutical into the object under examination. During the time-resolved acquisition of the molecular image data, advantageously, the patient is not repositioned and/or moved.

The acquired molecular image data can be used to determine the radiation dose of the radiopharmaceutical in the at least one target area and/or area at risk. To this end, the at least one target area and/or area at risk in the magnetic resonance image data can be transferred to the molecular image data. Consequently, the at least one target area and/or at least one area at risk in which the radiation dose is determined can be identified with reference to the segmentation in the magnetic resonance image data. This procedure is based on the consideration that the at least one target area and/or area at risk can typically be determined more accurately in the magnetic resonance image data than in the molecular image data since the magnetic resonance image data typically represents anatomical structures better than the molecular image data. One reason for this is, for example, the magnetic resonance image data typically exhibits a higher contrast between tissue located in the at least one target area and/or area at risk and surrounding tissue than the molecular image data.

The radiation dose can be determined in dependence on the activity in the molecular image data in the at least one target area and/or area at risk. In this case, a higher measured activity is indicative of a higher radiation dose. Use is made to the fact that the molecular image data directly represent a distribution of the radiopharmaceutical, which is also used to treat the object under examination. Alternatively, it is also conceivable for the molecular image data to be acquired by a radioactive tracer substance, which is different from the radiopharmaceutical. The radioactive tracer substance will then typically display similar accumulation behavior to that of the radiopharmaceutical thus enabling a conclusion to be drawn from the molecular image data acquired by the radioactive tracer substance regarding the radiation dose of the radiopharmaceutical. A procedure of this kind is described in one of the following sections. The determination of the radiation dose of the radiopharmaceutical with reference to the molecular image data includes an estimation of the radiation dose of the radiopharmaceutical.

The inventive procedure enables an efficient and reliable determination of the radiation dose of the radiopharmaceutical. The interplay between the magnetic resonance image data and the molecular image data is of decisive significance. The molecular image data can lead to a conclusion regarding the distribution of the radiopharmaceutical in the body of the object under examination, while the magnetic resonance image data can be used to determine the at least one target area and/or area at risk in which the radiation dose is to be determined. In this way, the radiation dose of the radiopharmaceutical can be determined in the correct regions, which are determined with reference to the magnetic resonance image data, with a high degree of accuracy due to the use of the molecular image data.

In an embodiment, the segmentation of the at least one target area and/or at least one area at risk is used to generate segmentation information, wherein the determination of the radiation dose is performed using the segmentation information. The segmentation information typically includes at least the information on the site in the body of the object under examination at which the at least one target area and/or area at risk is located. The segmentation information can be used to define the at least one target area and/or area at risk in the molecular image data. To this end, it may be necessary for the segmentation information to be adapted to match a scaling and/or a recording of the molecular image data. The determination of the radiation dose can then include the determination of an activity measured in the molecular image data in the at least one target area and/or area at risk identified with reference to the segmentation information. As already described, it is particularly advantageous to perform the segmentation of the at least one target area and/or area at risk for the determination of the radiation dose in the magnetic resonance image data since it is typically simpler to demarcate the at least one target area and/or area at risk from the environment in the magnetic resonance image data than in the molecular image data. Furthermore, selective highlighting of the at least one target area and/or area at risk in the magnetic resonance image data is possible using a dedicated contrast medium.

In another embodiment, the magnetic resonance image data and the molecular image data are acquired by operation of a combined imaging system, with the acquisitions occurring at least partially simultaneously. The combined imaging apparatus includes the magnetic resonance image data acquisition unit (scanner) and the molecular image data acquisition unit (scanner). The at least partially simultaneous acquisition of the magnetic resonance image data and the molecular image data means that at least one part of the magnetic resonance image data is acquired during at least one part of the duration of the acquisition of the molecular image data. The duration of the acquisition of the molecular image data can in this way at least partially overlap the duration of the acquisition of the molecular image data. It is also conceivable for the magnetic resonance image data and the molecular image data to be acquired completely simultaneously over the same examination period. Like the molecular image data, the magnetic resonance image data can be at least partially acquired following the commencement of the introduction of the radiopharmaceutical in the body of the object under examination. This is meaningful when, in an initial time of the introduction of the radiopharmaceutical during which the magnetic resonance image are acquired, no critical radiation dose is to be expected in the at least one area at risk. This makes it possible to reduce the entire recording time. It is also possible to significantly shorten the sequence of operations for determining the radiation dose since one single combined imaging apparatus is used for the acquisition of the magnetic resonance image data and the molecular image data. To this end, the combined imaging apparatus can be designed as a combined positron emission tomography magnetic resonance device (PET-MR device). It is also conceivable for the combined imaging apparatus to be a single photon emission tomography image data acquisition unit (SPECT-MR device) or another type of combined imaging apparatus that appears appropriate to those skilled in the art.

In another embodiment, the acquisition of the magnetic resonance image data is performed using a magnetic resonance sequence that is operated dependent on target tissue in the at least one target area and tissue at risk in the at least one area at risk, so that a contrast between the target area and the area at risk in the magnetic resonance image data lies above a specific threshold. In this case, the target tissue represents at least a part of the tissue of the object under examination located in the target area. In this case, the tissue at risk represents at least a part of the tissue of the object under examination located in the area at risk. It is advantageous for the magnetic resonance image data to have a first minimum contrast between the at least one target area and/or area at risk and the surrounding tissue. It is furthermore advantageous for the magnetic resonance image data to have a second minimum contrast between the at least one target area and/or the at least one area at risk. The first minimum contrast and/or second minimum contrast be can in particular 2:1, advantageously 4:1, most advantageously 8:1. This enables a particularly simple and accurate segmentation of the at least one target area and/or area at risk in the magnetic resonance image data. To ensure that the desired contrast is present in the magnetic resonance image data, the magnetic resonance sequence can be selected as dedicated.

In another embodiment, the determination of the radiation dose of the radiopharmaceutical in the at least one target area and/or at least one area at risk is performed at several points in time during the accumulation of the radiopharmaceutical. To this end, an activity of the radiopharmaceutical is determined continuously during the accumulation of the radiopharmaceutical with reference to the molecular image data, in particular using segmentation information generated from the magnetic resonance image data.

Typically, to this end, the acquisition of the molecular image data is commenced with or immediately after a start of the introduction of the radiopharmaceutical into the object under examination. However, it is also possible to acquire molecular reference image data before the introduction of the radiopharmaceutical into the object under examination. In this case, the duration of the accumulation of the radiopharmaceutical at least extends in particular at least over a duration of the introduction of the radiopharmaceutical into the object under examination. In order to determine the radiation dose at several time points, it is possible to acquire a number of molecular images by operation of the molecular image data acquisition unit at the several time points. Acquisition of dynamic molecular image data can be performed. The molecular image data is in particular embodied with time resolution. The radiation dose determined at the several time points can particularly advantageously be used to track the accumulation of the radiopharmaceutical in the at least one target area and/or area at risk. It is in principle also conceivable for the radiation dose determined with time resolution to be adapted for treatment of the object under examination by means of the radiopharmaceutical as described in one of the following sections.

In another embodiment, a first threshold for the radiation dose in the at least one target area and/or a second threshold for the radiation dose be defined in the at least one area at risk, wherein a comparison is made, for several points in time, between the radiation dose of the radiopharmaceutical determined in the at least one target area and/or at least one area at risk with the first threshold and/or the second threshold. This procedure is based on the consideration that typically a minimum radiation dose, which can correspond to the first threshold, is to be applied to the target area so that a desired effect of the radiopharmaceutical can take effect. There is also typically a maximum radiation dose, which can correspond to the second threshold, for the at least one area at risk. In this case, the maximum radiation dose should advantageously not be exceeded in order to enable unwanted damage to tissue located in the area at risk to be avoided. A comparison of the determined radiation dose with the first threshold and/or second threshold thus can provide particularly useful information. To this end, it is possible for a result of the comparison to be provided as an output.

In another embodiment, a radiation dose control signal is emitted when the radiation dose determined in the at least one target area reaches the first threshold and/or the radiation dose determined in the at least one area at risk reaches the second threshold. The radiation dose control signal can also be emitted only when the first threshold and/or second threshold is exceeded. The radiation dose control signal can include an actuation of an output interface, for example a monitor or a speaker. The output device can then emit information, for example warning information, for a user. Alternatively or additionally, the radiation dose control signal can include an actuation of a injection device, which introduces the radiopharmaceutical into the object under examination. For example, the radiation dose control signal can include an adaptation, for example stopping, of the introduction of the radiopharmaceutical into the object under examination. The radiation dose control signal can also include an actuation of the magnetic resonance image data acquisition unit and/or the molecular image data acquisition unit. For example, an acquisition of the magnetic resonance image data and/or the molecular image data can be adapted, for example stopped, in dependence on the radiation dose control signal. Hence, the radiation dose control signal enables a suitable reaction to the first threshold and/or of the second threshold being reached.

In another embodiment, a third threshold is defined for the ratio between the radiation dose in the at least one target area and the radiation dose in the at least one area at risk, wherein the ratio between the radiation dose of the radiopharmaceutical determined in the at least one target area and the radiation dose of the radiopharmaceutical determined in the at least one area at risk is compared with the third threshold at the several time points. This procedure is based on the consideration that a ratio between the radiation dose in the at least one target area and the radiation dose in the at least one area at risk is of interest. This enables a ratio to be established between an efficacy of the radiopharmaceutical in the at least one target area and damage to tissue located in the at least one area at risk by the radiopharmaceutical. A comparison of the determined radiation dose with the third threshold can, therefore, provide particularly useful information. Obviously it is also conceivable for the radiation dose to be compared with the third threshold additionally to the aforementioned first threshold and/or second threshold.

In another embodiment, a radiation dose control signal is emitted when the ratio between the radiation dose of the radiopharmaceutical determined in the at least one target area and the radiation dose of the radiopharmaceutical determined in the at least one area at risk reaches the third threshold. The radiation dose control signal can also only be output when the third threshold is exceeded. The radiation dose control signal can be embodied as described above. Hence, the radiation dose control signal enables a suitable reaction when the third threshold is reached.

In a further embodiment, the determination of the radiation dose of the radiopharmaceutical in the at least one target area and/or in the at least one area at risk is performed using a pharmacokinetic model. The pharmacokinetic model takes into account processes implemented by the radiopharmaceutical applied to the object under examination with the body of the object under examination. In this case, pharmacokinetic models as far as possible take account of the entirety of processes experienced by the radiopharmaceutical applied to the object under examination in the body of the patient. These processes can inter alia include absorption of the radiopharmaceutical, distribution of the radiopharmaceutical in the body, degradation of the radiopharmaceutical and excretion of the radiopharmaceutical. For example, the pharmacokinetic model can represent a delay in the accumulation of the radiopharmaceutical in the at least one target area and/or area at risk. Correspondingly, the radiation dose of the radiopharmaceutical can even be determined during a part of the accumulation time of the radiopharmaceutical. For example, the accumulation of the radiopharmaceutical in the at least one target area and/or area at risk can be modeled. In this case, the pharmacokinetic model is case in particular used additionally to the molecular image data for the determination of the radiation dose. This enables the radiation dose of the radiopharmaceutical in the at least one target area and/or area at risk to be determined even more accurately and/or efficiently.

It is also conceivable for a range of the radiation emitted by the radiopharmaceutical in a tissue of the at least one target area and/or area at risk to be taken into account during the determination of the radiation dose. This is advantageous when the radiopharmaceutical is a beta emitter and/or a gamma emitter.

In another embodiment, the acquired magnetic resonance image data include perfusion magnetic resonance image data, and the perfusion magnetic resonance image data are used to determine blood-flow information for the at least one target area and/or at least one area at risk. The determination of the radiation dose of the radiopharmaceutical in the at least one target area and/or in the at least one area at risk is performed using the blood-flow information. The perfusion magnetic resonance image data can describe the blood-flow in the at least one target area and/or area at risk. A strong blood-flow in the at least one target area and/or area at risk can in this case result in higher absorption of the radiopharmaceutical in the at least one target area and/or area at risk. For example, in this case, it is possible to take account of an arterial input function of the radiopharmaceutical during the determination of the radiation dose. In this way, the magnetic resonance image data can be used particularly advantageously, in addition to the generation of the segmentation information, to determine the radiation dose.

In a further embodiment, the molecular image data are acquired during the accumulation of a radioactive tracer substance in the at least one target area and/or the at least one area at risk, wherein the radioactive tracer substance has similar accumulation behavior to that of the radiopharmaceutical. The radioactive tracer substance has similar accumulation behavior in the at least one target area and/or area at risk as that of the radiopharmaceutical. The radioactive tracer substance and the radiopharmaceutical can have substantially the same pharmacokinetic and/or pharmacological properties, in particular with respect to the accumulation in the at least one target area and/or area at risk. For example, the radioactive tracer substance can be coupled to the same binding substance, such as to the same antibody and/or the same peptide as the radiopharmaceutical. For example, the radioactive tracer substance and the radiopharmaceutical can belong to the same substance class and/or have a similar polarity and/or a similar molecular weight. In addition, the pharmacokinetic properties of the radioactive tracer substance and the radiopharmaceutical can also be pharmacologically the same with respect to absorption or accumulation in the at least one target area and/or area at risk or with respect to absorption in the patient's bloodstream, distribution in a body of the object under examination, metabolization in a tissue of the at least one target area and/or area at risk or of degradation in the at least one target area and/or area at risk. For example, the distribution, perfusion or diffusion of the radioactive tracer substance can be substantially the same as that of the radiopharmaceutical. This can enable conclusions to be made regarding the distribution of the radiopharmaceutical from the distribution of the radioactive tracer substance. The radioactive tracer substance can be administered to the object under examination at the same time as the radiopharmaceutical. Alternatively, the radioactive tracer substance can be administered to the object under examination in advance of the radiopharmaceutical. Advantageously, a lower radiation effective amount of the radioactive tracer substance than the radiation effective amount of the radiopharmaceutical can be administered. For example, the dose ratio between the radiopharmaceutical and the radioactive tracer substance can be greater than 5:1, advantageously greater than 10:1, most advantageously greater than 20:1. The radiation dose of the radiopharmaceutical can be determined such that a determined radiation dose of the radioactive tracer substance and/or an activity of the radioactive tracer substance in the at least one target area and/or area at risk is extrapolated to the radiation dose of the radiopharmaceutical. This procedure is advantageous when the radiation of the radioactive tracer substance can be detected more efficiently than the radiation of the radiopharmaceutical by means of the molecular image data acquisition unit. For example, the radiation of the radiopharmaceutical cannot be detected by the molecular image data acquisition unit. This can be the case when the radiopharmaceutical is an alpha emitter. The radioactive tracer substance is then advantageously a beta emitter, for example as a beta-plus emitter, or is a gamma emitter.

In a further embodiment, during the accumulation of the radiopharmaceutical, further magnetic resonance image data are acquired. The magnetic resonance image data and the further magnetic resonance image data are used to determine movement information characterizing a movement of the object under examination between the acquisition of the magnetic resonance image data and the further magnetic resonance image data and the segmentation of the at least one target area and/or at least one area at risk is adapted with reference to the movement information for a determination of the radiation dose of the radiopharmaceutical. The movement information can be determined from a rigid or elastic registration of the further magnetic resonance image data to the magnetic resonance image data. In this way, the segmentation information determined by the magnetic resonance image data can be dynamically adapted with reference to the movement information. In this way, the at least one target area and/or area at risk in which the radiation dose of the radiopharmaceutical is to be determined can be adapted dynamically to the movement of the object under examination. This enables a more accurate determination of the radiation dose of the radiopharmaceutical.

The radiation dose determining unit according to the invention has a magnetic resonance image data acquisition unit (scanner), a molecular image data acquisition unit and a computing unit with a segmenting unit and a dose determining module, wherein the radiation dose determining unit is embodied to carry out a method according to the invention.

Hence, the radiation dose determining unit is designed to carry out a method for determining a radiation dose of a radiopharmaceutical. The magnetic resonance image data acquisition unit is operated for the acquisition of magnetic resonance image data of an object under examination. The segmenting unit is configured for the segmentation of at least one target area and/or at least one area at risk for accumulation of the radiopharmaceutical in the magnetic resonance image data. The molecular image data acquisition unit is operated for the acquisition of molecular image data of the object under examination during the accumulation of the radiopharmaceutical in the at least one target area and/or the at least one area at risk. The dose determining module is configured to determine a radiation dose of the radiopharmaceutical in the at least one target area and/or the at least one area at risk using the molecular image data.

In an embodiment of the radiation dose determining unit, the computer has a comparator, which is configured to produce a comparative value characterizing a result of a comparison of at least one radiation dose determined by means of the dose determining module with at least one threshold. The at least one threshold can include the above described first threshold and/or second threshold and/or third threshold. The comparative value generated by the comparator can be used to generate the radiation dose control signal from a control signal generation unit in the comparison unit.

The system according to the invention has a radiation dose determining unit according to the invention with the comparator, and an injection apparatus for the injection of a radiopharmaceutical, wherein the injection apparatus has an injection control unit and, for purposes of data exchange, is connected to the comparator such that the injection control unit is configured to control the injection of the radiopharmaceutical with reference to the comparative value transmitted by the comparator to the injection control unit. In particular, the control signal generating unit of the comparator transmits a radiation dose control signal generated with reference to the comparative value to the injection control unit. Then, the injection control unit can, for example, interrupt the injection of the radiopharmaceutical in dependence on the received comparative value and/or radiation dose control signal. In this way, the injection of the radiopharmaceutical can be advantageously adapted to the measured radiation dose of the radiopharmaceutical in the at least one target area and/or area at risk.

The combined imaging system according to the invention has a magnetic resonance image data acquisition unit (scanner), a molecular image data acquisition unit (scanner), and a radiation dose determining unit according to the invention. The radiation dose determining unit is configured to send control signals to the combined imaging system and/or to receive or process control signals in order to carry out the method according to the invention. The radiation dose determining unit can be integrated in the combined imaging system. The radiation dose determining unit can also be installed separately from the combined imaging system. The radiation dose determining unit can be connected to the combined imaging system. The acquisition of the magnetic resonance image data can include the recording of the magnetic resonance image data by the molecular image data acquisition unit of the combined imaging system. The acquisition of the molecular image data can include a recording of the molecular image data by the molecular image data acquisition unit of the combined imaging system. The magnetic resonance image data and the molecular image data can then be transferred to the radiation dose determining unit for further processing.

The invention also encompasses a non-transitory, computer-readable data storage medium that can be loaded directly into a memory of a programmable computer of a radiation dose determining unit and has program code (i.e. it is encoded with programming instructions) in order to carry out a method according to the invention when the instructions are executed in the computer of the radiation dose determining unit. This enables the method according to the invention to be carried out quickly, identically repeatably and robustly. In this case, the computer must have the requisite peripherals such as an appropriate user memory, an appropriate graphics card or an appropriate logic unit so that the respective method steps can be carried out efficiently.

Examples of electronically readable data media are a DVD, a magnetic tape or a USB stick on which electronically readable control information, in particular software (see above) is stored.

The advantages of the radiation dose determining unit according to the invention, the system according to the invention, the combined imaging system according to the invention and the data storage medium according to the invention substantially correspond to the advantages of the method according to the invention, as explained in detail above. Any features, advantages or alternative embodiments mentioned above also are applicable to the other aspects of the invention. The functional features of the method are embodied by corresponding modules, in particular hardware modules.

### BRIEF DESCRIPTION OF THE DRAWINGS

FIG. 1 schematically shows an embodiment of a system according to the invention and a combined imaging system according to the invention.

FIG. 2 is a flowchart of a first embodiment of the method according to the invention.

FIG. 3 is a flowchart of a second embodiment of the method according to the invention.

FIG. 4 shows an example of segmentation of a target area and a area at risk in magnetic resonance image data.

FIG. 5 shows an example of a procedure for the comparison of radiation doses determined with a first threshold and a second threshold.

### DESCRIPTION OF THE PREFERRED EMBODIMENTS

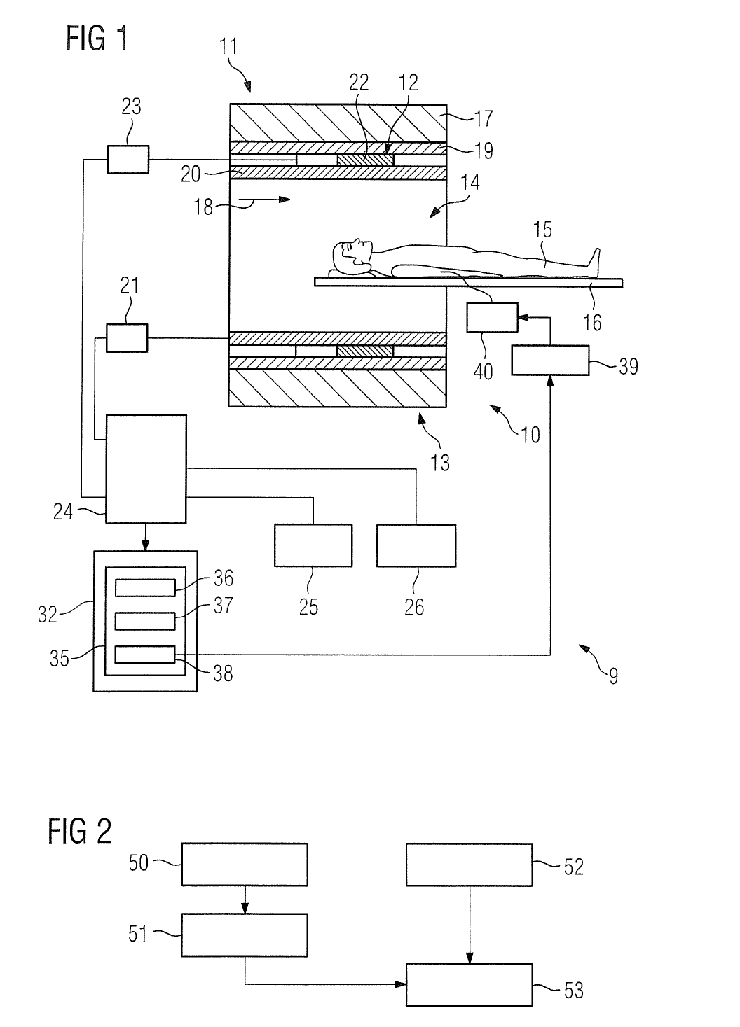

FIG. 1 shows an embodiment of a system according to the invention 9 and a combined imaging system according to the invention 10 in a schematic view.

The combined imaging system 10 shown is embodied is shown as an example as a magnetic resonance/PET device 10. A medical imaging system 10 according to the invention generally has a magnetic resonance image data acquisition unit (scanner), a molecular image data acquisition unit (scanner), and a radiation dose determining unit (processor). The combined imaging system can also be embodied as a magnetic resonance/SPECT device.

The shown magnetic resonance PET device 10 has a magnetic resonance device 11, which forms the magnetic resonance image data acquisition unit and a positron emission tomography device 12 (PET device 12), which forms the molecular image data acquisition unit. The magnetic resonance device 11 has a scanner 13 and a patient receiving area 14 surrounded by the scanner 13 to receive an object under examination 15, in particular a patient 15, wherein the patient receiving area 14 is surrounded in a circumferential direction by the scanner 13 in a cylindrical shape. The patient 15 can be moved into the patient receiving area 14 by a patient support 16 of the magnetic resonance/PET device 10. To this end, the patient support 16 is arranged movably inside the patient receiving area 14.

The scanner 13 has a basic field magnet 17, which, during the operation of the magnetic resonance device 11, is configured to generate a strong and constant basic magnetic field 18. The scanner 13 further has a gradient coil unit 19 to generate magnetic field gradients, which are operated for spatial encoding during imaging. The scanner 13 also has a radio-frequency (RF) antenna unit 20, which in the case shown is designed as a body coil permanently integrated in the scanner 13, which is provided to excite nuclear spins in the patient 15 so as to cause the spins to deviate from the polarization that is established in the basic magnetic field 18 generated by the basic field magnet 17. The radio-frequency antenna unit 20 is furthermore provided to receive magnetic resonance signals that result from the excited spins.

To control the basic field magnet 17, the gradient coil unit 19, and the radio-frequency antenna unit 20, the magnetic resonance/PET device 10, in particular the magnetic resonance device 11, has a magnetic resonance control computer 21. The magnetic resonance control computer 21 provides central control for the magnetic resonance device 11, such as for the performance of a predetermined imaging gradient echo sequence. To this end, the magnetic resonance control computer 21 has a gradient control unit (not individually shown) and a radio-frequency antenna control unit (not individually shown). The magnetic resonance control computer 21 also has an evaluation unit (not individually shown) for the evaluation of magnetic resonance image data.

The magnetic resonance device 11 can have further components that are usually present in magnetic resonance devices. The general mode of operation of a magnetic resonance apparatus is known to those skilled in the art so that a detailed description of the general components is not necessary herein.

The PET device 12 has multiple positron emission tomography detector modules 22 (PET detector modules 22) that are arranged in a ring so as to surround the patient receiving area 14 in the circumferential direction. Each PET detector module 22 has multiple positron emission tomography detector elements (PET detector elements) (not shown in detail) arranged to form a PET detector array, namely a scintillation detector array with scintillation crystals, for example LSO crystals. Each PET detector module 22 also has a photodiode array, for example an avalanche photodiode array (APD array) downstream of the scintillation detector array inside each PET detector module 22.

The PET detector modules 22 are used to acquire photon pairs that result from the annihilation of a positron with an electron in the patient 15. Trajectories of the two photons form an angle of 180°. The two photons each have an energy of 511 keV. In this case, the positron is emitted by a radiopharmaceutical that has been administered to the patient 15 by injection. Upon passing through material, photons produced during the annihilation can be attenuated, and the probability of such attenuation is determined by the path length through the material and the attenuation coefficients of the material along the path. Accordingly, when evaluating the PET signals, it is necessary to correct these signals with respect to the attenuation due to material components situated in the beam path.

In addition, each PET detector module 22 has detector electronics with an electric amplifier circuit and further electronic components (not individually shown). To control the detector electronics and the PET detector modules 22, the magnetic resonance/PET device 10, in particular the PET device 12, has a PET control computer 23. The PET control computer 23 provides central control for the PET device 12. In addition, the PET control computer 23 has an evaluation unit for evaluation of PET data.

The PET device 12 depicted can have further components that are usually present in devices. The general mode of operation of a PET computers is also known to those skilled in the art known so that a detailed description of the general components is not necessary herein.

The magnetic resonance/PET device 10 also has a central control computer 24, which, for example, matches the acquisition and/or evaluation of magnetic resonance image data and PET image data to one another. The control computer 24 can be a central system control computer. Control information such as imaging parameters, and reconstructed image data can be displayed on a display monitor 25, for example on at least one monitor screen, of the magnetic resonance PET device 10 for a user. In addition, the magnetic resonance PET device 10 has an input interface 26, via which a user can enter information and/or parameters during a measuring process. The control computer 24 can include the magnetic resonance control computer 21 and/or the PET control computer 23 and/or the display monitor 25 and/or the input interface 26.

The system 9 shown further has a radiation dose determining unit 32 according to the invention and an injection apparatus 40 for the injection of a radiopharmaceutical. The injection apparatus 40 has an injection control processor 39. The radiation dose determining unit 32 is simultaneously embodied as part of the magnetic resonance PET device 10, but this is not mandatory. It is also conceivable for the radiation dose determining unit 32 to be separate from the magnetic resonance PET device 10 and for only data recorded by the magnetic resonance PET device 10 to be acquired.

The radiation dose determining unit 32 as shown has a computer 35 with a segmenting unit 36 and a dose determining module 37. In this way, the radiation dose determining unit 32 shown together with the magnetic resonance PET device 10 is configured to carry out the method according to the invention for determining a radiation dose of a radiopharmaceutical.

For solely carrying out the method according to the invention, the radiation dose determining unit 32 will include a magnetic resonance image data acquisition unit (not depicted) and a molecular image data acquisition unit (not depicted). The magnetic resonance image data acquisition unit then acquires magnetic resonance image data, which was recorded by the magnetic resonance device 11 of the magnetic resonance PET device 10. The molecular image data acquisition unit then acquires molecular image data, which was recorded by means of the PET device 12 of the magnetic resonance PET device 10. To this end, the magnetic resonance image data acquisition unit and the molecular image data acquisition unit are advantageously connected to the control device 24 of the magnetic resonance PET device 10 for the purposes of data exchange.

In the case depicted, the computer 35 of the radiation dose determining unit 32 includes a comparator 38 that emits a radiation dose control signal, characterizing a result of a comparison of at least one radiation dose determined by the dose determining module 37 with at least one threshold.

In this way, the injection control unit 39 is connected, for data exchange, to the comparator 38 so that the injection control unit 39 is configured to control the injection of the radiopharmaceutical with reference to the radiation dose control signal transmitted from the comparison unit 38 to the injection control computer 39.

FIG. 2 is a flowchart of a first embodiment of the method according to the invention for determining a radiation dose of a radiopharmaceutical.

In a first method step 50, magnetic resonance image data of an object under examination 15 are acquired by means of the magnetic resonance image data acquisition unit. In this case, it is conceivable for the magnetic resonance image data to be recorded by a magnetic resonance device, for example the magnetic resonance device 11 of the magnetic resonance/PET device 10. Alternatively, it is possible to acquire magnetic resonance image data that has already been recorded by the magnetic resonance image data acquisition unit of the radiation dose determining unit 32. To this end, the radiation dose determining unit 32 is, for example, able to access a database on which the magnetic resonance image data is stored.

In a further method step 51, at least one target area and/or at least one area at risk for accumulation of the radiopharmaceutical in the magnetic resonance image data is segmented by the segmenting unit 36. The segmentation can in this case be performed automatically and/or manually. It is conceivable for the segmentation to be based on information regarding the organ of the object under examination 15 in which the radiopharmaceutical typically accumulates.

In a further method step 52, molecular image data of the object under examination 15 are acquired by the molecular image data acquisition unit during the accumulation of the radiopharmaceutical in the at least one target area and/or the at least one area at risk. The molecular image data can be recorded by means of a molecular imaging unit, for example the PET device 12 of the magnetic resonance/PET device 10, during the accumulation of the radiopharmaceutical. Alternatively, it is also conceivable for the molecular image data acquisition unit of the radiation dose determining unit 32 to acquire molecular image data recorded during the accumulation of the radiopharmaceutical. To this end, the molecular image data can, for example, be loaded from a database.

In a further method step 53, a radiation dose of the radiopharmaceutical in the at least one target area and/or the at least one area at risk using the molecular image data is determined by means of the dose determining module 37. The radiation dose of the radiopharmaceutical can in this case be, for example, determined with reference to a specific activity of the radiopharmaceutical determined in the at least one target area and/or area at risk by means of the molecular image data. The radiation dose is in particular determined using the segmentation of the at least one target area and/or area at risk.

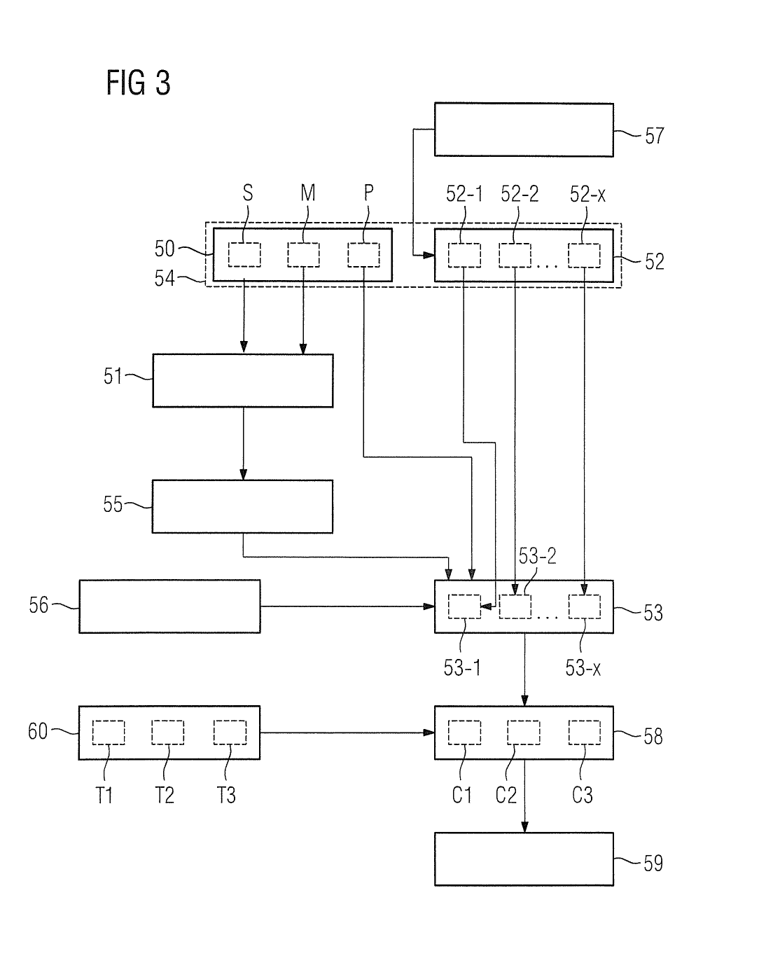

FIG. 3 is a flowchart of a second embodiment of the method according to the invention for determining a radiation dose of a radiopharmaceutical.

The following description is substantially restricted to the differences from the exemplary embodiment in FIG. 2, wherein reference is made to the description of the exemplary embodiment in FIG. 2b with respect to method steps that remain the same. Method steps that substantially remain the same are in principle given the same reference characters.

The embodiment of the method according to the invention shown in FIG. 3 includes the method steps 50, 51, 52, 53 of the first embodiment of the method according to the invention shown in FIG. 2. The embodiment of the method according to the invention shown in FIG. 3 also has additional method steps and substeps. An alternative method to that in FIG. 3, which has only some of the additional method steps and/or substeps shown in FIG. 2, is also conceivable. The method alternative to FIG. 3 can also have additional method steps and/or substeps.

In the case shown, the acquisition of the magnetic resonance image data in the further method step 50 and the acquisition of the molecular image data in the further method step 52 are performed by a combined imaging system at least partially simultaneously in a further method step 54. The combined medical imaging system, which is, for example, embodied as a magnetic resonance/PET device 10 shown in FIG. 1, includes the magnetic resonance image data acquisition unit and molecular image data acquisition unit required for this. To this end, the two image data acquisition units are in particular integrated in the combined medical imaging system so that the simultaneous acquisition of the magnetic resonance image data and the molecular image data is possible from an at least partially overlapping area under examination.

The acquisition of the magnetic resonance image data in the further method step 50 is performed using a magnetic resonance sequence S, which is adapted to a target tissue in the at least one target area and a tissue at risk in the at least one area at risk such that a contrast between the target tissue and the tissue at risk in the magnetic resonance image data is above a specific threshold. For example, a STIR-TSE magnetic resonance sequence S has been found to be advantageous. The STIR-TSE magnetic resonance sequence can depict bone marrow lesions, for example. Diffusion-weighted magnetic resonance sequences S can be used to depict organs, such as the spleen. Magnetic resonance sequences S with the suppression and/or saturation of water tissue can be used to depict fatty bone marrow. It is also possible to use other magnetic resonance sequences S that appear appropriate to those skilled in the art. It is also conceivable for a contrast medium to be administered to the object under examination 15 for the acquisition of the magnetic resonance image data. For example, a liver-specific contrast medium, such as Primovist, enables a demarcation of liver tissue from liver metastases.

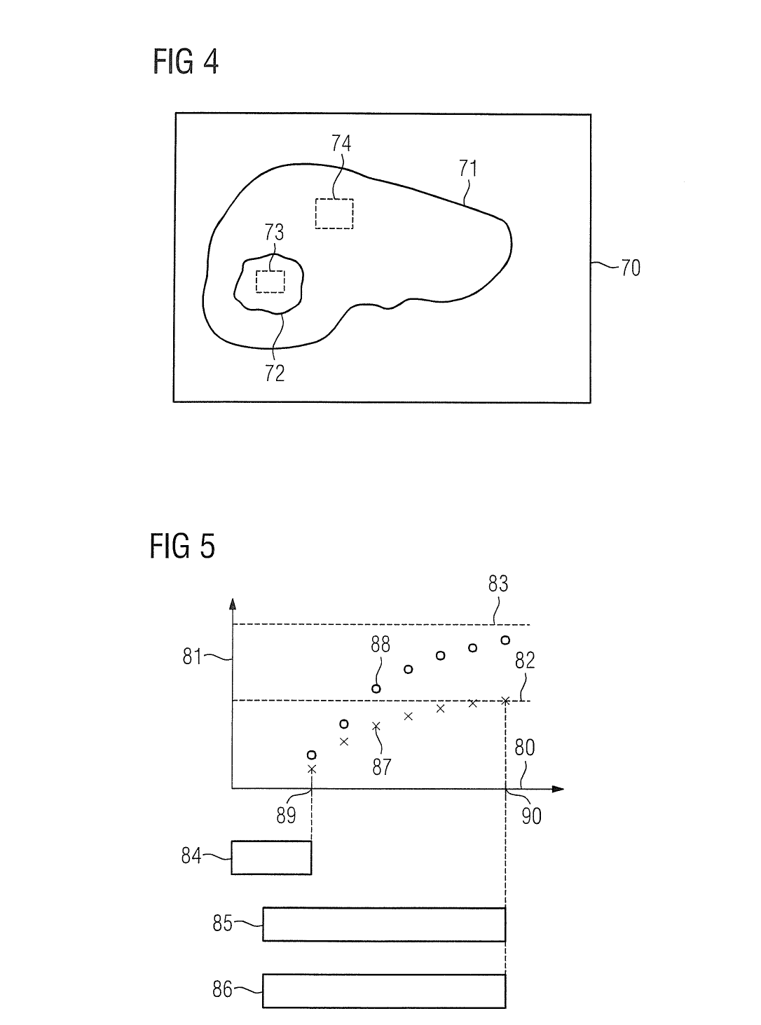

A suitable choice of the magnetic resonance sequence S enables a particularly simple segmentation of the at least one target area and/or area at risk in the further method step 51. FIG. 4 depicts an exemplary segmentation of a target area and an area at risk in magnetic resonance image data. FIG. 4 shows in this case a slice 70 of a magnetic resonance image depicting a liver 71 of the object under examination 15. The magnetic resonance image was recorded by operation of a suitable magnetic resonance sequence, for example using a contrast medium such as Primovist. A liver metastasis 72 has been identified in the liver. In the further method step 51, the liver metastasis 72 has been segmented as a target area for irradiation by the radiopharmaceutical. In the further method step 51, the liver 71 without the liver metastasis 72 was segmented as an area at risk for irradiation by the radiopharmaceutical. This procedure is based on the consideration that, during irradiation by the radiopharmaceutical, in particular the liver metastasis 72 is to be irradiated by means of an adequate radiation dose, while the liver 71 itself does not receive any toxic radiation dose. Consequently, it is possible to define a target area-measuring region 73 in the segmented liver metastasis 72 in which a radiation dose of the radiopharmaceutical can be determined for extrapolation to the radiation dose of the target area in the further method step 53. Consequently, it is possible to define an area at risk-measuring region 74 in the segmented liver 71 in which a radiation dose of the radiopharmaceutical can be determined for extrapolation to the radiation dose of the area at risk in the further method step 53. In the case shown in FIG. 4, the area at risk surrounds the target area completely by way of example. However, this is not mandatory. It is also conceivable for the area at risk to be arranged separately from the target area. Neither does the area at risk necessarily have to bound the target area.

The segmentation of the at least one target area and/or at least one area at risk in the further method step 51 is used to generate segmentation information by the segmenting unit 36 in a further method step 55. The radiation dose in the further method step 53 is then determined using the segmentation information. To this end, the segmentation information can be transmitted from the segmenting unit 36 to the dose determining module 37. The segmentation information can define the spatial region of the molecular image data in which the radiation dose is to be determined. For example, the segmentation information can define the at least one target area and/or area at risk in the molecular image data.

Optionally, in a further method step 56, it is possible to provide a pharmacokinetics model by means of the dose determining module 37. The radiation dose of the radiopharmaceutical in the at least one target area and/or in the at least one area at risk in the further method step 53 can then be determined using the pharmacokinetic model.

It is also possible for the magnetic resonance image data acquired in the further method step 50 to include perfusion magnetic resonance image data P, wherein the perfusion magnetic resonance image data P is used to determine blood-flow information for the at least one target area and/or at least one area at risk, wherein the determination of the radiation dose of the radiopharmaceutical in the at least one target area and/or in the at least one area at risk in the further method step 53 is performed using the blood-flow information.

It is further possible for a radioactive tracer substance to be introduced into the object under examination 15 in a further method step 57. In this case, the radioactive tracer substance is advantageously to a large extent administered to the object under examination 15 at the same time as the radiopharmaceutical. The radioactive tracer substance has similar accumulation behavior to that of the radiopharmaceutical. The molecular image data can then be acquired in the further method step 52 during the accumulation of the radioactive tracer substance in the at least one target area and/or the at least one area at risk. This procedure is in particular then appropriate when the radiopharmaceutical cannot be identified directly in the molecular image data. Then, it is possible to make conclusions regarding the distribution of the radiopharmaceutical from the distribution of the radioactive tracer substance and hence determine the radiation dose of the radiopharmaceutical.

It is also optionally possible to take account of a movement of the object under examination 15 during the accumulation of the radiopharmaceutical during the determination of the radiation dose. To this end, during the accumulation of the radiopharmaceutical, further magnetic resonance image data M can be acquired in the further method step 50, wherein the magnetic resonance image data and the further magnetic resonance image data M are used to determine movement information characterizing a movement of the object under examination 15 between the acquisition of the magnetic resonance image data and the further magnetic resonance image data and the segmentation of the at least one target area and/or at least one area at risk is adapted in the further method step 51 with reference to the movement information for a determination of the radiation dose of the radiopharmaceutical. In this case, it is conceivable for initially a first radiation dose to be determined before a movement of the object under examination 15 using a segmentation information compiled on the basis of the magnetic resonance image data. Then, a second radiation dose can be determined following a movement of the object under examination 15 using segmentation information compiled on the basis of the further magnetic resonance image data M.

In the case shown in FIG. 3, the radiation dose of the radiopharmaceutical in the at least one target area and/or at least one area at risk is determined at several time points during the accumulation of the radiopharmaceutical. To this end, in the case shown, the molecular image data includes multiple molecular image data sets 52-1, 52-2, . . . , 52-x, which are acquired in the further method step 52 at the several time points during the accumulation of the radiopharmaceutical. For example, a first molecular image data set 52-1 of the multiple molecular image data sets 52-1, 52-2, . . . , 52-x is acquired at a first time point, a second molecular image data set 52-2 of the multiple of molecular image data sets 52-1, 52-2, . . . , 52-x at a second time point, etc. The multiple of molecular image data sets 52-1, 52-2, . . . , 52-x can then be used to determine the various radiation doses of the radiopharmaceutical as a function of time. In the further method step 53, then multiple dose values 53-1, 53-2, . . . , 53-x of the radiopharmaceutical are determined in the at least one target area and/or area at risk. The first molecular image data set 52-1 can in this case be based on the determination of a first dose value 53-1 of the multiple of dose values 53-1, 53-2, . . . , 53-x, the second molecular image data set 52-2 can in this case be based on the determination of a second dose value 53-2 of the multiple of dose values 53-1, 53-2, . . . , 53-x, etc.

In a further method step 60, at least one threshold T1, T2, T3 for the radiation dose in the at least one target area and/or area at risk can be determined by means of the comparison unit 38. For example, a first threshold T1 can be defined for the radiation dose in the at least one target area. Alternatively or additionally, a second threshold T2 can be defined for the radiation dose in the at least one area at risk. Alternatively or additionally, a third threshold T3 can be defined for a ratio between the radiation dose in the at least one target area and the radiation dose in the at least one area at risk. The at least one threshold T1, T2, T3 can be defined automatically and/or manually.

In a further method step 58, a first comparison C1 of the radiation dose of the radiopharmaceutical determined in the at least one target area with the first threshold T1 at the several time points can be performed by the comparator 38. Alternatively or additionally, a second comparison C2 of the radiation dose of the radiopharmaceutical determined in the at least one area at risk with the second threshold T2 at the several time points can be performed by means of the comparison unit 38. A procedure of this kind is illustrated by way of example in FIG. 5.

It is also conceivable in a third comparison C3, which can be performed alternatively or additionally to the first comparison C1 and/or second comparison C2, for the ratio between the radiation dose of the radiopharmaceutical determined in the at least one target area and the radiation dose of the radiopharmaceutical determined in the at least one area at risk at the several time points to be compared with the third threshold T3. This procedure can be appropriate in the case of irradiation of a bone metastasis as a target area by means of the radiopharmaceutical. Part of the radiopharmaceutical can accumulate in the spleen of the object under examination 15 as an area at risk. It is then possible to define that a multiple, for example twenty times the radiation dose, should be present in the bone metastasis than that in the spleen. This ratio between the radiation dose in the bone metastasis and the spleen can be checked at the several points in time. It is thereby possible to control the infusion of the radiopharmaceutical. The ratio between the radiation doses can in this case in particular also be checked with reference to a partial dose, for example ten percent, of the radiopharmaceutical administered to the object under examination 15.

In a further method step 59, the comparator 38 can emit a radiation dose control signal when the radiation dose determined in the at least one target area reaches the first threshold T1 and/or the radiation dose determined in the at least one area at risk reaches the second threshold T2. The radiation dose control signal can also be output when the ratio between the radiation dose of the radiopharmaceutical determined in the at least one target area and the radiation dose of the radiopharmaceutical determined in the at least one area at risk reaches the third threshold T3. Hence, the radiation dose control signal can be output with reference to the result of the first comparison C1 and/or the second comparison C2 and/or the third comparison C3. The radiation dose control signal can for example be transmitted from the comparator 38 to the display monitor 26 so that information can be displayed to a user on the display unit 26. Advantageously, the radiation dose control signal is transmitted from the comparator 38 to the injection control computer 39. The injection control computer 39 can then control the injection apparatus 40 with reference to the radiation dose control signal received. For example, the injection control computer 39 can cause the injection of the radiopharmaceutical into the object under examination 15 to be interrupted with reference to the received radiation dose control signal.

The method steps of the method according to the invention in depicted in FIGS. 2-3 are carried out by the computer. To this end, the computer includes the necessary software and/or computer programs, which are stored in a storage unit of the computing unit. The software and/or computer program has programming instructions (code) configured to carry out the method according to the invention when the computer program and/or the software in the computer are executed by means of a processor unit of the computer.

FIG. 5 shows an exemplary procedure for the comparison of determined radiation doses with a first threshold and a second threshold. Reference is made to the fact that the procedure depicted in FIG. 5 only represents one possibility for carrying out the method according to the invention. Consequently, FIG. 5 is only intended for purposes of illustration. For example, modifications to the thresholds and time ranges are conceivable. It is also obviously possible to measure another temporal course of the radiation doses.

On a vertical dose axis 81, a determined radiation dose over a period of time is plotted on a horizontal time axis 80. A second threshold 82 for the area at risk radiation dose 87 determined in an area at risk and a first threshold 83 for the target area radiation dose 88 determined in a target area are shown. Furthermore, a temporal course of the area at risk radiation dose 87 determined in the area at risk is identified by crosses. Furthermore, a temporal course of the target area radiation dose 88 determined in the target area determined is indicated by dots in the diagram.

Plotted below the horizontal time axis 80, is a first time range 84 during which an acquisition of magnetic resonance image data of the object under examination 15 is performed. Furthermore, a second time range 85 is identified during which the radiopharmaceutical is introduced into the object under examination 15. A third time range 86 indicates a duration of the acquisition of the molecular image data.

As shown in FIG. 5, the acquisition of the magnetic resonance image data and the molecular image data is performed partially simultaneously since the first time range 84 and the third time range 86 partially overlap. This can result in shortening of the measuring time, but does mandatorily have to be the case. In the case depicted in FIG. 5, the introduction of the radiopharmaceutical and the acquisition of the molecular image data are performed simultaneously for example.

When the acquisition of the magnetic resonance image data is completed, the magnetic resonance image data for the target area and the area at risk can be segmented, such as, for example, depicted in FIG. 4. The segmented target area and area at risk and the acquired molecular image data can then be used to determine the radiation dose in the target area and the area at risk. In this way, the conclusion of the first time range 84 at a first time point 89 enables a determination of a first measured value for the radiation dose in the target area and a first measured value for the radiation dose in the area at risk.

In this way, in the case shown in FIG. 5, the introduction of the radiopharmaceutical into the object under examination 15 is controlled such that the introduction of the radiopharmaceutical is interrupted when the target area radiation dose 88 determined reaches the first threshold 83 or when the area at risk radiation dose 87 determined reaches the second threshold 82. In the case shown in FIG. 5, as an example the area at risk radiation dose 87 first reaches the second threshold 82 at a second point in time 90. In this way, according to the exemplary criteria, the introduction of the radiopharmaceutical into the object under examination 15 is interrupted at the second point in time 90. The acquisition of the molecular image data can possibly also be stopped at the second point in time.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for determining a radiation dose of a radiopharmaceutical, comprising:
operating a magnetic resonance (MR) scanner while an object is situated therein, to acquire MR image data from the object;
providing the MR image data to a processor and, in said processor, executing a segmentation algorithm to segment, in the MR image data, at least one area selected from the group consisting of a target area and an area that is at risk for accumulation of said radiopharmaceutical;
operating a molecular data scanner, while the object is situated therein, to acquire molecular image data from said at least one area during accumulation of said radiopharmaceutical in said at least one area; and
providing the molecular image data to said processor and, in said processor, automatically determining a radiation dose of said pharmaceutical in said at least one area, using said molecular image data, and making an electrical signal representing said radiation dose available as an output from said processor.

2. A method as claimed in claim 1 comprising, in said processor, deriving segmentation information from the segmentation of said at least one area, and determining said radiation dose also using said segmentation information.

3. A method as claimed in claim 1 comprising operating said MR scanner to acquire said MR image data at least partially simultaneously with operation of said molecular data scanner to acquire said molecular image data.

4. A method as claimed in claim 1 comprising operating said MR scanner to acquire said MR image data by execution of an MR data acquisition sequence that is matched to target tissue in said at least one area, with a contrast between said target area and said area at risk in said MR image data being above a predetermined threshold.

5. A method as claimed in claim 1 comprising determining said radiation dose of said pharmaceutical area in said at least one area at each of a plurality of points in time during accumulation of said radiopharmaceutical.

6. A method as claimed in claim 1 comprising, in said processor, executing said segmentation algorithm to segment, in said MR image data, each of a target area and an area at risk for accumulation of the radiopharmaceutical, and defining a first threshold for said radiation dose in said target region and a second threshold for said radiation dose in said area at risk, and determining said radiation dose of said radiopharmaceutical in each of said target area and said area at risk, and comparing said radiation dose of said radiopharmaceutical in said target area with said first threshold to obtain a first comparison result, and comparing said radiation dose of said radiopharmaceutical in said area at risk with said second threshold to obtain a second comparison result.

7. A method as claimed in claim 6 comprising emitting a radiation dose control signal either when said radiation dose in said target area reaches said first threshold, as indicated by said first comparison result, or when said radiation dose in said area at risk reaches said second threshold, as indicated by said second comparison result.

8. A method as claimed in claim 6 comprising, in said processor, determining a ratio between the radiation dose of said radiopharmaceutical in said target area and the radiation dose of said radiopharmaceutical in said area at risk, and defining a third threshold for said ratio in said processor, and comparing said ratio to said third threshold to obtain a third comparison result.

9. A method as claimed in claim 8 comprising emitting a radiation dose control signal when said ratio reaches said third threshold, as indicated by said third comparison result.

10. A method as claimed in claim 1 comprising determining said radiation dose of said radiopharmaceutical in said at least one area by using a pharmacokinetic model in said processor.

11. A method as claimed in claim 1 comprising operating said MR scanner to acquire said MR image data as perfusion MR image data and, in said processor, using said perfusion magnetic resonance image data to determine blood flow information of said at least one area, and determining said radiation dose of said radiopharmaceutical in said at least one area using said blood flow information.

12. A method as claimed in claim 1 comprising operating said molecular data scanner to acquire said molecular image data during accumulation of a radioactive tracer substance in said at least one area, said radioactive tracer substance having an accumulation behavior corresponding to an accumulation behavior of said radiopharmaceutical.

13. A method as claimed in claim 1 comprising operating said MR scanner to acquire further MR image data from the object during said accumulation of said radiopharmaceutical and providing said further magnetic resonance image data to said processor and, in said processor, determining movement information from said MR image data and said further MR image data that describes a movement of the object between a time of acquisition of said MR image data and a time of acquisition of said further MR image data, and adapting the segmentation of said at least one area dependent on said movement information when determining said radiation dose of said radiopharmaceutical in said at least one area.

14. A radiation dose determining apparatus comprising:
a magnetic resonance (MR) scanner;
a molecular data scanner;
a control computer configured to operate said MR scanner while an object is situated therein, to acquire MR image data from the object;
a processor provided with the MR image data, said processor being configured to execute a segmentation algorithm to segment, in the MR image data, at least one area selected from the group consisting of a target area and an area that is at risk for accumulation of said radiopharmaceutical;
said control computer being configured to operate said molecular data scanner, while the object is situated therein, to acquire molecular image data from said at least one area during accumulation of said radiopharmaceutical in said at least one area; and
said processor being provided with the molecular image data, and said processor being configured to automatically determine a radiation dose of said pharmaceutical in said at least one area, using said molecular image data, and to make an electrical signal representing said radiation dose available as an output from said processor.

15. A radiation dose determining apparatus as claimed in claim 14 wherein said computer comprises a comparator configured to produce a comparison result by comparing at least one radiation dose with at least one threshold.

16. A radiation dose determining system, comprising:
a magnetic resonance (MR) scanner;
a control computer configured to operate said MR scanner while an object is situated therein, to acquire MR image data from the object;
a processor provided with the MR image data, said processor being configured to execute a segmentation algorithm to segment, in the MR image data, at least one area selected from the group consisting of a target area and an area that is at risk for accumulation of said radiopharmaceutical;
a molecular data scanner;
said control computer being configured to operate said molecular data scanner, while the object is situated therein, to acquire molecular image data from said at least one area during accumulation of said radiopharmaceutical in said at least one area;
said processor being provided with the molecular image data to said processor and, in said processor, automatically determining a radiation dose of said pharmaceutical in said at least one area, using said molecular image data, said processor comprising a comparator configured to emit a comparative value that represents a result of a comparison of said radiation dose with at least one threshold, and said processor being configured to make an electrical signal representing said comparative value available as an output from said processor; and
an injection apparatus configured to inject said radiopharmaceutical into the object, said injection apparatus comprising an injection control processor in data exchange with said comparator to receive said comparative value therefrom, said injection control processor being configured to control injection of said radiopharmaceutical dependent on said comparative value.

17. An imaging system comprising:
a magnetic resonance (MR) scanner and a molecular data scanner combined in a common unitary housing;
a control computer configured to operate said MR scanner while an object is situated therein, to acquire MR image data from the object;
a processor provided with the MR image data, said processor being configured to execute a segmentation algorithm to segment, in the MR image data, at least one area selected from the group consisting of a target area and an area that is at risk for accumulation of said radiopharmaceutical;
said control computer being configured to operate said molecular data scanner, while the object is situated therein, to acquire molecular image data from said at least one area during accumulation of said radiopharmaceutical in said at least one area; and
said processor being provided with the molecular image data, and said processor being configured to automatically determine a radiation dose of said pharmaceutical in said at least one area, using said molecular image data, and to make an electrical signal representing said radiation dose available as an output from said processor.

18. A non-transitory, computer-readable data storage medium encoded with programming instructions, said storage medium being loaded into a control and processing computer system of an imaging apparatus that comprises a magnetic resonance (MR) scanner and a molecular data scanner, said programming instructions causing said control and processing computer system to:
operate the MR scanner while an object is situated therein, to acquire MR image data from the object;
execute a segmentation algorithm to segment, in the MR image data, at least one area selected from the group consisting of a target area and an area that is at risk for accumulation of said radiopharmaceutical;
operate the molecular data scanner, while the object is situated therein, to acquire molecular image data from said at least one area during accumulation of said radiopharmaceutical in said at least one area; and
automatically determine a radiation dose of said pharmaceutical in said at least one area, using said molecular image data, and make an electrical signal representing said radiation dose available as an output from said processor.
